(19) United States
(12) Patent Application Publication
(10) Pub. No.: US 2011/0071075 A1
(43) Pub. Date: Mar. 24, 2011

(54) PROMOTER FOR BICARBONATE SECRETION IN GASTROINTESTINAL TRACT

(76) Inventors: Koji Takeuchi, Kyoto-shi (JP); Eitaro Aihara, Kyoto-shi (JP); Eiji Nakamura, Kawasaki-shi (JP); Tetsuo Yano, Kawasaki-shi (JP); Mai Hasumura, Kawasaki-shi (JP); Hisayuki Uneyama, Kawasaki-shi (JP)

(21) Appl. No.: 12/888,479

(22) Filed: Sep. 23, 2010

Related U.S. Application Data
(63) Continuation of application No. PCT/JP2009/055769, filed on Mar. 24, 2009.

(30) Foreign Application Priority Data
Mar. 24, 2008 (JP) 2008-076362

(51) Int. Cl. A61K 38/00; A61P 3/04; A61K 31/195; A61K 31/135; A61P 1/04

(52) U.S. Cl. 514/4.9; 514/13.2; 514/561; 514/649

(57) ABSTRACT

A substance is described which can prevent or treat a disease associated with the secretion of an acid. A calcium receptor activator is the active ingredient in a composition which promotes bicarbonate secretion in a gastrointestinal tract. Examples of the calcium receptor activator include γ-Glu-X-Gly, wherein X represents an amino acid or an amino acid derivative, γ-Glu-Val-Y, wherein Y represents an amino acid or an amino acid derivative, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH2, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me).

This application is a continuation under 35 U.S.C. §120 of PCT Patent Application No. PCT/JP2009/055769, filed Mar. 24, 2009, which claims priority under 35 U.S.C. §119 to Japanese Patent Application No. 2008-076362, filed on Mar. 24, 2008, which are incorporated in their entireties by reference.

### BACKGROUND OF THE INVENTION

1. Field of the Invention

The present invention relates to a promoter of bicarbonate secretion in the gastrointestinal tract. The promoter of bicarbonate secretion can be utilized in a composition which can be used to enhance bicarbonate secretion in the prophylaxis or treatment of gastrointestinal dysfunctions, such as acid secretion-related diseases. The promoter can also be used in a food that is effective for the prophylaxis and improvement of gastrointestinal dysfunctions.

2. Brief Description of the Related Art

Gastrointestinal conditions related to excess secretion of acid are frequently observed in clinical practice. These conditions include gastric and duodenal ulcers, ulcers caused by ingestion of non-steroidal anti-inflammatory drugs (NSAID), gastro-esophageal reflux disease (GERD), and non-erosive reflux disease (NERD).

Peptic ulcers such as gastric and duodenal ulcers can form as a result of exposure of the gastric and duodenal mucosa to an excess amount of acids or digestive enzymes, or which develop as a result of the breakdown of the mucosal defense mechanism. Peptic ulcers are considered to be caused by, for example, (1) infection with Helicobacter pylori, (2) use of non-steroidal anti-inflammatory drugs and steroidal drugs, and (3) stress, drinking, and smoking. Therapeutic drugs for peptic ulcers are broadly classified as those which inhibit aggressive factors, and those which enhance defensive factors. Examples of drugs which inhibit aggressive factors include a histamine H2 receptor inhibitor and a proton pump inhibitor, both of which each inhibit acid secretion. Examples of drugs which enhance defensive factors include ecabet and L-glutamine, both of which function by coating the gastric mucosa. It is also known that patients positive for Helicobacter pylori obtain a high therapeutic effect by eliminating the bacteria (The merck manual of medical information second home edition, online version (researched on Mar. 13, 2008), Internet: mmh banyu.co.jp/mmhe2j/sec09/ch121/ch121c.html).

Gastrointestinal disorders may be induced by low-dose aspirin, which is the therapeutic drug of choice for antiplatelet therapy, and NSAID, which is prescribed to the elderly for the purpose of, for example, treating the pain associated with osteoarthritis. In recent years, the development and use of double balloon and capsule endoscopes have increasingly led to the discovery of NSAID-induced ulcers in the small intestine. A prostaglandin formulation, a proton pump inhibitor (PPI), and the like, are used as therapeutic drugs for such NSAID-induced small intestinal mucosal damage, but therapeutic effects thereof are said to be less than satisfactory. Furthermore, it has been reported that NSAID-induced ulcers induce intestinal hemorrhage, and 17,000 people per year die from this condition in the United States (Nippon Rinsho 2007; Vol. 65(10): pp. 1749-1758).

GERD is a disease in which the esophageal mucosa is damaged by the reflux of an acid or a digestive enzyme into the esophagus due to a disorder of the lower esophageal sphincter, which acts to prevent reflux. However, it is known that, unlike peptic ulcers, the elimination of Helicobacter pylori exacerbates this condition. Furthermore, NERD, which causes reflux symptoms such as heartburn but does not cause endoscopically-observed mucosal damage, has also started to attract attention in recent years (Nippon Rinsho 2007; Vol. 65(5): pp. 797-801, 841-845).

An histamine H2 receptor inhibitor, a proton pump inhibitor, and the like, are mainly used as therapeutic drugs for these acid secretion-related diseases. The histamine H2 receptor inhibitor inhibits gastric acid secretion during the night, and does not inhibit gastric acid secretion after meals. On the other hand, the proton pump inhibitor inhibits gastric acid secretion caused by histamine, gastrin, and acetylcholine, and shows higher effectiveness than the histamine H2 receptor inhibitor. However, when acid secretion inhibitors are administered for a long period of time, the growth of the gastric mucosa is observed; and furthermore, the rebound of acid secretion occurs after stopping administration, resulting in the relapse of ulcers and inflammations. Furthermore, these drugs must be carefully administered to patients with renal or hepatic dysfunction. From the foregoing, drugs which are able to both reduce aggressive factors and enhance defensive factors in treating diseases related to acid secretion with less adverse effects even in long-term administration are desirable.

Meanwhile, appetite regulators, such as fenfluramine, phentermine, sibutramine, mazindol, and the like, which act on the central nervous system are known, but may cause adverse effects such as dry mouth, constipation, sweating, and palpitation. Therefore, there is a demand for an appetite regulator with less adverse effects (“Konnichi no Chiryouyaku 2001, Explanations and Handbook”, edited by Yutaka Mizushima, Nankodo Inc., p. 928).

Calcium receptor is also called Calcium Sensing Receptor (hereinafter, also referred to as “CaSR”). The calcium receptor was cloned from bovine thyroid in 1993, and found to be a G-protein coupled seven-transmembrane receptor (G-protein coupled receptor; GPCR) capable of sensing extracellular calcium (Calcium; Ca²⁺) (Brown, E. M. et al. 1993. Nature Vol. 366: pp. 575-580). By sensing extracellular Ca²⁺, the calcium receptor is able to alter the intracellular Ca²⁺ concentration, thereby regulating the production of, for example, hormones involved in the metabolic regulation of Ca²⁺, such as parathyroid hormone. In recent years, it has been found that cinacalcet (CCT), which is a calcium receptor agonist, is able to inhibit parathyroid hormone secretion by acting on the calcium receptor in the parathyroid, resulting in an increase in the Ca²⁺ sensitivity of the calcium receptor (Cohen, A. and Silverberg, S. J. 2002. Current Opinion in Pharmacology 2: pp. 734-739). Furthermore, it has been found that the calcium receptor is expressed in the kidney, brain, thyroid, bone, and gastrointestinal tract (McLamon, S. J. and Riccardi, D. et al. 2002. European Journal of Pharmacology Vol. 447: pp. 271-278), as well as in G cells and parietal cells in the stomach, and is able to promote gastrin and gastric acid secretion (Ray, J. M. et al. 1997. Journal of Clinical Investigation Vol. 99: pp. 2328-2333 and Cheng, I. et al. 1999. Gastroenterology 1999; Vol. 116: pp. 118-126). Furthermore, the calcium receptor is expressed in the large intestine, where it acts to regulate water secretion (Cheng, S. X. et al. 2002. The American Journal of Physiology-Gastroinstinal and Liver Physiology Vol. 283: pp. G240-G250). In view of the foregoing, it has been reported that a calcium receptor agonist can be effective for upper and lower gastrointestinal diseases (WO 2006/123725 pamphlet).

It has been demonstrated that a calcium receptor agonist regulates acid secretion, and its action of inhibiting alkalization in parietal cells under histamine stimulation is comparable to that of omeprazole, which is a proton pump inhibitor (Geibel, J. P. et al. 2001. Journal of Biological Chemistry Vol. 276: pp. 39549-39552).

As described above, the ability of the calcium receptor agonist to inhibit acid secretion, which is one of the aggressive factors for the gastrointestinal mucosa, has started to become clear, but the effect on defensive factors still remains unknown.

### SUMMARY OF THE INVENTION

Aspects of the present invention include providing a medicament capable of preventing or treating diseases related to acid secretion. The medicament is effective for mucosal injuries induced by an excessive acid secretion caused by gastrointestinal dysfunction, diseases, or drugs.

A calcium receptor activator which promotes bicarbonate secretion in the stomach and duodenum is described. The inventors have also confirmed that the calcium receptor activator inhibits histamine-induced gastric acid secretion. In addition, a technology for isolating specific cells of the stomach is described, and based on this technology, the calcium receptor has been found to be expressed in gastric D cells. In addition, the calcium receptor activator has been found to promote somatostatin secretion in gastric D cells. Based on these results, novel applications are described for prophylactic or therapeutic drugs for various diseases related to acid secretion, in which a composition containing the calcium receptor activator is administered or ingested to enhance defensive factors in the stomach and duodenum.

In addition, the calcium receptor activator has been found to promote somatostatin secretion in gastric D cells, while glutamic acid inhibits somatostatin secretion. Furthermore, because somatostatin acts on the digestive organs, such as the stomach, small intestine, large intestine, pancreas, and the like, and inhibits the secretion of appetite-regulating hormones and the like, the calcium receptor activator can be used in combination with glutamic acid as an appetite regulator.

It is an aspect of the present invention to provide a method for promoting bicarbonate secretion in the gastrointestinal tract, comprising administering a composition comprising calcium receptor activator to a subject in need of the promotion of bicarbonate secretion in the gastrointestinal tract.

It is a further aspect of the present invention to provide the method as described above, wherein the calcium receptor activator is a peptide.

It is a further aspect of the present invention to provide the method as described above, wherein the peptide is selected from the group consisting of γ-Glu-X-Gly, γ-Glu-Val-Y, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me); wherein X is selected from the group consisting of an amino acid and an amino acid derivative; and Y is selected from the group consisting of an amino acid and an amino acid derivative.

It is a further aspect of the present invention to provide the method as described above, wherein X is selected from the group consisting of Cys, Cys(SNO), Cys(S-allyl), Gly, Cys(S-Me), Abu, and Ser; and Y is selected from the group consisting of Gly, Val, Glu, Lys, Phe, Ser, Pro, Arg, Asp, Met, Thr, His, Orn, Asn, Cys, and Gln.

It is a further aspect of the present invention to provide the method as described above, wherein the calcium receptor activator is selected from the group consisting of:

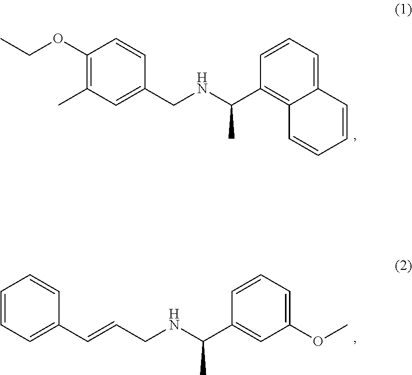

and salts thereof.

It is a further aspect of the present invention to provide the method as described above, wherein the peptide is γ-Glu-Val-Gly.

It is a further aspect of the present invention to provide the method as described above, wherein the calcium receptor activator is cinacalcet.

It is an aspect of the present invention to provide a method for preventing or treating a disease related to acid secretion comprising administering a composition comprising calcium receptor activator to a subject in need of the prophylaxis of treatment of said disease.

It is a further aspect of the present invention to provide the method as described above, wherein the disease is selected from the group consisting of gastric ulcer, duodenal ulcer, non-steroidal anti-inflammatory drug-induced ulcer, gastro-esophageal reflux disease, and non-erosive reflux disease.

It is an aspect of the present invention to provide a method for preventing or treating a non-steroidal anti-inflammatory drug-induced ulcer comprising administering composition comprising γ-Glu-Val-Gly or cinacalcet to a subject in need thereof. It is a further aspect of the present invention to provide the method as described above, wherein the calcium receptor activator is administered in an amount of 0.001 to 10 g/kg body weight per day.

It is a further aspect of the present invention to provide a method for regulating appetite comprising alternatively administering a calcium receptor activator or the appetite stimulator to a subject in need of appetite regulation.

It is a further aspect of the present invention to provide the method as described above, wherein the appetite stimulator is sodium L-glutamate.

### BRIEF DESCRIPTION OF THE DRAWINGS

FIGS. 1A and 1B illustrate the influences of cinacalcet on bicarbonate secretion in the stomach and duodenum, respectively.

FIG. 2 illustrates the influence of cinacalcet on histamine-induced gastric acid secretion.

FIGS. 3A and 3B illustrate the expression amounts of somatostatin and CaSR, respectively, in cell fractions of rat gastric mucosa.

FIG. 4 illustrates the amount of somatostatin secretion when the fractionated gastric mucosal D cells are treated with a CaSR agonist.

FIG. 5 illustrates histamine-induced gastric acid secretion when using a combination of cinacalcet and a somatostatin receptor 2 inhibitor.

FIG. 6 illustrates the effect of cinacalcet on NSAID-induced enteritis.

FIG. 7 illustrates the effect of γ-EVG on NSAID-induced enteritis.

FIG. 8 illustrates the influence of γ-EVG on the amount of bicarbonate secretion in the duodenum.

FIG. 9 illustrates the amount of somatostatin secretion fractionated gastric mucosal D cells are treated with a CaSR agonist and sodium glutamate.

### DESCRIPTION OF THE PREFERRED EMBODIMENTS

A composition which promotes bicarbonate secretion in the gastrointestinal tract is described. This composition can contain a calcium receptor activator as the active ingredient.

The term “calcium receptor” can refer to a receptor that is known as the Calcium Sensing Receptor (CaSR) and belongs to class C of the seven-transmembrane receptors. The term “CaSR activator” can include a substance that regulates the functions of CaSR-expressing cells by binding and activating the CaSR (hereinafter, also referred to as “CaSR agonist”), and a substance that functions to extend the CaSR activity by binding and activating the CaSR (hereinafter, also referred to as “CaSR modulator”). Furthermore, the term “CaSR activation” can mean that a ligand binds to a calcium receptor to activate a guanine nucleotide binding protein, and thereby transduces a signal. In addition, transduction of this signal by the calcium receptor can be referred to as “CaSR activity”.

A calcium receptor activator can be obtained by the screening methods described below, for example, but the methods are not limited to these.

A CaSR agonist and modulator can be obtained via screening by determining the presence or absence of CaSR activation by a test substance by using CaSR-expressing cells.

The presence or absence of CaSR activation can be determined by measuring the amount of a substance (ligand) that binds to the CaSR, a substance that inhibits a reaction with a signal for regulating the CaSR activity, a substance (such as a second messenger) that transduces a signal generated by the binding of a ligand to the CaSR, or the like. For example, CaSR activation can be measured by detecting a second messenger generated by the binding of a ligand such as Ca²⁺ to CaSR. Furthermore, CaSR activation can also be measured by using a radiolabeled, known ligand, and measuring the binding of the radiolabeled ligand to the CaSR.

The CaSR bound to a ligand can act on a GTP-binding protein (also referred to as a G protein, such as Gi or Gq) to control various cell functions via a second messenger such as cAMP. In particular, Gq activation increases the intracellular calcium concentration. Furthermore, downstream of the increase in intracellular calcium concentration in the signal transduction pathway, functions are acutely regulated both through the activation of intracellular enzymes such as calmodulin, protein kinase C, and adenylate cyclase, and through the phosphorylation of cytoplasmic proteins/cell membrane proteins. The activation of intracellular enzymes can alter channel function in the cell membrane. Thus, the presence or absence of CaSR activation by a test substance can be detected by bringing the test substance into contact with CaSR-expressing cells, and observing G protein activation using a pre-determined intracellular calcium concentration, the amount of intracellular cAMP, the channel function (such as the extracellular proton production amount), the amount of gastrointestinal hormone secretion, or the like, as an indicator.

CaSR-expressing cells that can be used for screening can be, for example, cells derived from animals including mammals such as mice, rats, hamsters, guinea pigs, rabbits, dogs, monkeys, and human beings, and avian species such as chickens. For example, gastrointestinal hormone-producing cells derived from the above-described animals can be used. Furthermore, the origin of the CaSR is not particularly limited, and, for example, can be a CaSR derived from the above-described animals. Specifically, the human CaSR encoded by the human CaSR gene registered under GenBank Accession No NM_{—}000388 can be used. It should be noted that the CaSR is not limited to the protein encoded by the gene having the above-described sequence, and can be a protein encoded by a gene having a homology of 60% or more, or in other examples 80% or more, 90% or more, and 98% or more, to the above-described sequence as long as the gene encodes a protein having the CaSR function. The GPRC6A receptor or 5.24 receptor is also known as a subtype of the CaSR, and can be used.

The test substance that can be used in screening can be a known or novel compound, and examples include nucleic acids, saccharides, lipids, proteins, peptides, organic low-molecular-weight compounds, compound libraries prepared by using combinatorial chemistry technology, random peptide libraries prepared by solid-phase synthesis or a phage display method, and natural ingredients derived from microorganisms, animals and plants, marine organisms or the like.

A first screening method (hereinafter, also referred to as “method A”) can include, for example, the following steps (a), (b), and (c):

(a) a step of bringing CaSR-expressing cells into contact with a test substance;

(b) a step of measuring the G protein activation in the cells brought into contact with the test substance, and comparing such activation with the activation in control cells not brought into contact with the test substance; and

(c) a step of selecting a substance capable of activating CaSR based on the comparison results in step (b).

In step (a), the CaSR-expressing cells are kept in contact with the test substance, which can occur in a culture medium. The culture medium can be appropriately selected depending on the kind of cells to be used and the like.

In step (b), firstly, the activation of G proteins in the CaSR-expressing cells in the presence of the test substance can be evaluated. Next, this activation is compared with the activation in the absence of the test substance. Here, examples of an indicator which can be used to measure the activation of G proteins include the intracellular calcium concentration, the amount of intracellular cAMP, the amount of extracellular protein, the amount of secretion of an intracellular gastrointestinal hormone, such as somatostatin, and the like.

In step (c), the comparison of the activations can be conducted based on, for example, the presence or absence of a significant difference. As a result of the evaluation, if the activation is increased or extended in the presence of the test substance as compared to that in the absence of the test substance, the test substance can be assessed as a CaSR agonist.

Also, when screening for a CaSR modulator in step (a), the test substance and a CaSR agonist can be brought into contact with the CaSR-expressing cells; in the step (b), the activation of G proteins when the CaSR agonist is brought into contact with the cells in the presence of a test substance is compared with the activation of G proteins when the CaSR agonist is brought into contact with the cells in the absence of the test substance; and in the step (c), a substance which extends the activation of G proteins can be selected as a CaSR modulator.

A second screening method for a CaSR agonist or modulator can include, for example, the following steps (a), (b), and (c):

(a) a step of bringing a test substance and a ligand acting on CaSR into contact with CaSR-expressing cells;

(b) a step of measuring the amount of the ligand bound to the cell membrane of the cells, and comparing the amount of the ligand with the amount of the ligand in control cells not brought into contact with the test substance; and

(c) a step of selecting a substance capable of activating CaSR based on the comparison results in step (b).

In step (a) of the second screening method, the CaSR-expressing cells are kept in contact with the test substance and the ligand acting on CaSR, which can occur in a culture medium. The culture medium can be appropriately selected depending on the kind of cells to be used and the like.

In step (b), firstly, the amount of the ligand bound to the cell membrane of the CaSR-expressing cells in the presence of the test substance can be evaluated. Next, the amount of the ligand is compared with the amount of the ligand in the absence of the test substance. The amount of the ligand bound can be measured, for example, by using a radio-labeled ligand or the like.

In step (c), the comparison of the amounts of the ligand is conducted based on, for example, the presence or absence of a significant difference. As a result of the evaluation, if the amount of the ligand bound is decreased in the presence of the test substance as compared to that in the absence of the test substance, the test substance can be assessed as a CaSR agonist or a CaSR modulator.

In addition, substances that cause a decrease in the amount of bound ligand as determined by screening method A can be confirmed as CaSR agonists.

The ligand acting on CaSR is not particularly limited, and examples include Ca²⁺, cinacalcet, and the like.

Hereinafter, specific methods (1) to (6) for detecting a substance capable of improving gastrointestinal dysfunctions by using CaSR-expressing cells (cells having a functional CaSR) are exemplified.

(1) A Method Including the Following Steps (a), (b), and (c):

(a) a step of bringing CaSR-expressing cells into contact with a test substance for a certain period of time;

(b) a step of measuring somatostatin secreted from the cells brought into contact with the test substance, and comparing the secreted amount with the secreted amount in control cells not brought into contact with the test substance; and

(c) a step of selecting a substance capable of activating CaSR based on the comparison results in step (b).

(2) A Method Including the Following Steps (a), (b), and (c):

(a) a step of bringing CaSR-expressing cells containing a calcium sensitive dye (such as Fura-2, Indo-1, or Fluo-3) into contact with a test substance for a certain period of time;

(b) a step of measuring the fluorescence intensity (intracellular calcium concentration) in the cells brought into contact with the test substance, and comparing such an intensity with the intensity in control cells not brought into contact with the test substance; and

(c) a step of selecting a substance capable of activating the CaSR based on the comparison results in step (b).

Here, in step (a) of the above-described screening method, the CaSR-expressing cells can be gastrointestinal hormone-producing cells that express the CaSR receptor. For example, a substance can be tested for CaSR activation by measuring the alteration in the fluorescence intensity (intracellular calcium concentration) when gastrointestinal hormone-producing cells containing a calcium-sensitive dye are brought into contact with the test substance for a certain period of time. When screening for a CaSR modulator, the test substance and a CaSR agonist can be brought into contact with CaSR-expressing cells containing a calcium-sensitive dye, such as Fura-2, Indo-1, or Fluo-3.

In step (b) of the above-described screening method, whether or not the fluorescence intensity (intracellular calcium concentration) in CaSR-expressing cells varies in the presence of the test substance can be evaluated by comparing the fluorescence intensity (intracellular calcium concentration) with the fluorescence intensity (intracellular calcium concentration) in the absence of the test substance. The fluorescence intensity can be measured by a known method. When screening for a CaSR modulator, the fluorescence intensity when a CaSR agonist is brought into contact with the cells in the presence of the test substance can be compared with the fluorescence intensity when the CaSR agonist is brought into contact with the cells in the absence of the test substance.

In step (c) of the above-described screening method, the fluorescence intensities can be compared based on, for example, the presence or absence of a significant difference. As a result of the evaluation of the fluorescence intensity, if it can be confirmed that the intracellular calcium concentration increases, the test substance can be assessed as a CaSR agonist. When screening for a CaSR modulator, a large variation in the fluorescence intensity can indicate that the substance is a CaSR modulator.

(3) A Method Including the Following Steps (a), (b), and (c):

(a) a step of bringing CaSR-expressing cells into contact with a test substance for a certain period of time;

(b) a step of measuring the cAMP amount in the cells brought into contact with the test substance, and comparing the cAMP amount with the cAMP amount in control cells not brought into contact with the test substance; and

(c) a step of selecting a substance capable of activating CaSR based on the comparison results in the above-described (b).

The cAMP amount can be measured with a commercially available assay kit.

In step (a) of the above-described screening method, when screening for a CaSR modulator, the test substance and a CaSR agonist can be brought into contact with the CaSR-expressing cells.

In step (b) of the above-described screening method, when screening for a CaSR modulator, the amount of cAMP produced when a CaSR agonist is brought into contact with the cells in the presence of the test substance can be compared with the amount of cAMP when the CaSR agonist is brought into contact with the cells in the absence of the test substance.

In step (c) of the above-described screening method, the cAMP amounts can be compared based on, for example, the presence or absence of a significant difference. As a result of the evaluation of the cAMP amount, if it can be confirmed that the cAMP amount increases, the test substance can be assessed as a substance capable of activating CaSR. When screening for a CaSR modulator, a large increase in the cAMP amount indicates that the substance is a CaSR modulator.

(4) A Method Including the Following Steps (a), (b), and (c):

(a) a step of bringing a test substance and a known ligand (such as Ca²⁺ or cinacalcet) acting on CaSR into contact with CaSR-expressing cells for a certain period of time;

(b) a step of measuring the amount of the ligand bound to the cell membrane of the cells, and comparing this amount of the ligand with the amount of the ligand in control cells not brought into contact with the test substance; and

(c) a step of selecting a substance capable of activating CaSR based on the comparison results in the above-described step (b).

The amount of the known ligand can be measured by radio-labeling a part of the substances, and determining the amount of radioactivity bound to the cell membrane.

In step (c) of the above-described screening method, the amounts of the ligand can be compared based on, for example, the presence or absence of a significant difference. As a result of the evaluation of the amount of the ligand, if it can be confirmed that the amount of the ligand bound decreases, the test substance can be assessed as a CaSR agonist or modulator.

(5) A Method Including the Following Steps (a), (b), and (c):

(a) a step of bringing CaSR-expressing cells containing a cAMP sensitive fluorescent protein (such as FlCRhR) into contact with a test substance for a certain period of time;

(b) a step of measuring the fluorescence intensity (intracellular cAMP concentration) in the cells brought into contact with the test substance, and comparing this intensity with the intensity in control cells not brought into contact with the test substance; and

(c) a step of selecting a substance capable of activating CaSR based on the comparison results in the above-described step (b).

Here, the CaSR-expressing cells can be gastrointestinal hormone-producing cells which express CaSR.

In step (a) of the above-described screening method, when screening for a CaSR modulator, the test substance and a CaSR agonist can be brought into contact with the CaSR-expressing cells containing a cAMP sensitive fluorescent protein (such as FlCRhR).

In step (b) of the above-described screening method, when screening for a CaSR modulator, the fluorescence intensity (intracellular cAMP concentration) when a CaSR agonist is brought into contact with the cells in the presence of the test substance can be compared with the fluorescence intensity (intracellular cAMP concentration) when the CaSR agonist is brought into contact with the cells in the absence of the test substance.

In step (c) of the above-described screening method, the fluorescence intensities can be compared based on, for example, the presence or absence of a significant difference. As a result of the evaluation of the fluorescence intensity, if it can be confirmed that the fluorescence intensity increases, the test substance can be assessed as a CaSR agonist. When screening for a CaSR modulator, a large increase in the fluorescence intensity can indicate that the substance is a CaSR modulator.

(6) A Method Including the Following Steps (a), (b), and (c):

(a) a step of bringing CaSR-expressing cells into contact with a test substance for a certain period of time;

(b) a step of measuring the amount of extracellular proton production in the cells brought into contact with the test substance, and comparing this proton production amount with the extracellular proton production amount in control cells not brought into contact with the test substance; and

(c) a step of selecting a substance capable of activating CaSR based on the comparison results in the above-described (b).

Here, the CaSR-expressing cells can be gastrointestinal hormone-producing cells which express CaSR. For example, a target substance can be detected by measuring the extracellular proton production amount when gastrointestinal hormone cells which express CaSR are brought into contact with a CaSR agonist and a test substance for a certain period of time, and using the proton production amount as the indicator. The proton production amount is measured with a cytosensor.

In step (a) of the above-described screening method, when screening for a CaSR modulator, the test substance and a CaSR agonist can be brought into contact with the CaSR-expressing cells.

In step (b) of the above-described screening method, when screening for a CaSR modulator, the extracellular proton production amount when a CaSR agonist is brought into contact with the cells in the presence of the test substance can be compared with the extracellular proton production amount when the CaSR agonist is brought into contact with the cells in the absence of the test substance.

In step (c) of the above-described screening method, the proton production amounts can be compared based on, for example, the presence or absence of a significant difference. As a result of the evaluation of the proton production amount, if it can be confirmed that the extracellular proton production amount increases, the test substance can be assessed as a CaSR agonist. When screening for a CaSR modulator, a large increase in the extracellular proton production amount can indicates that the substance is CaSR modulator.

(7) A Method Including the Following Steps (a), (b), and (c):

(a) a step of bringing CaSR-expressing cells into contact with a test substance for a certain period of time;

(b) a step of measuring the amount of gastrointestinal hormone secretion in the cells which is in contact with the test substance, and comparing the amount of gastrointestinal hormone secretion with that of the gastrointestinal hormone secretion in control cells without the test substance; and

(c) a step of selecting a substance capable of activating CaSR based on the comparison results in the above-described (b).

Here, the CaSR-expressing cells can be gastrointestinal hormone-producing cells which express CaSR. For example, the amount of the gastrointestinal hormone secretion can be measured when gastrointestinal hormone-producing cells which express CaSR are brought into contact with a CaSR agonist and a test substance for a certain period of time, and using the amount of the gastrointestinal hormone secretion as the indicator. The amount of gastrointestinal hormone secretion can be measured by using a commercially available assay kit.

In step (a) of the above-described screening method, when screening for a CaSR modulator, the test substance and a CaSR agonist can be brought into contact with the CaSR-expressing cells.

In step (b) of the above-described screening method, in the case of screening a CaSR modulator, the amount of gastrointestinal hormone secretion when a CaSR agonist is brought into contact with the cells in the presence of the test substance can be compared with the amount of gastrointestinal hormone secretion when the CaSR agonist is brought into contact with the cells in the absence of the test substance.

In step (c) of the above-described screening method, the amount of gastrointestinal hormone secretion can be compared based on, for example, the presence or absence of a significant difference. As a result of the evaluation of the gastrointestinal hormone secretion amount, if it can be confirmed that the gastrointestinal hormone secretion amount varies, the test substance can be assessed as a CaSR agonist. When screening for a CaSR modulator, a large increase in the variation range of the gastrointestinal hormone secretion amount can indicate that the substance is a CaSR modulator.

The CaSR activator (CaSR agonist or modulator) obtained as above can be confirmed to be able to promote bicarbonate secretion in the gastrointestinal tract. Promotion of bicarbonate secretion can be confirmed in accordance with, for example, the methods of Aihara et al. (Aihara, E. et al. 2005. J. Pharmacol. Exp. Ther. 315: 423-432), Kagawa et al. (Kagawa, S. et al. 2003. Digestive Diseases and Sciences 48: 1850-1856), and those described in the Examples.

Specific examples of the CaSR agonist include cations each having two or more valences such as calcium and gadolinium; amino acids such as phenylalanine and tryptophan; various peptides such as γ-Glu-X-Gly (X represents an amino acid or an amino acid derivative), γ-Glu-Val-Y (Y represents an amino acid or an amino acid derivative), γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me); basic peptides such as polylysine; polyamines such as spermine and spermidine; proteins such as protamine; and various low-molecular-weight compounds such as cinacalcet and the compound represented by the chemical formula (1):

(R)—N-[(4-ethoxy-3-methylphenyl)methyl]-1-(1-naphthyl)ethylamine) or

the compound represented by the chemical formula (2):

(R)—N-(3-phenylprop-2-enyl)-1-(3-methoxyphenyl)ethylamine).

It should be noted that each of amino acids which constitute a peptide is in the L-form unless otherwise stated. Herein, examples of the amino acids include: a neutral amino acid such as Gly, Ala, Val, Leu, Ile, Ser, Thr, Cys, Met, Asn, Gln, Pro, or Hyp; an acidic amino acid such as Asp or Glu; a basic amino acid such as Lys, Arg, or His; an aromatic amino acid such as Phe, Tyr, or Trp; and homoserine, citrulline, ornithine, α-aminobutyric acid, norvaline, norleucine, and taurine.

Abbreviations for amino group residues can mean the following amino acids.

(1) Gly: Glycine

(2) Ala: Alanine

(3) Val: Valine

(4) Leu: Leucine

(5) Ile: Isoleucine

(6) Met: Methionine

(7) Phe: Phenylalanine

(8) Tyr: Tyrosine

(9) Trp: Tryptophan

(10) His: Histidine

(11) Lys: Lysine

(12) Arg: Arginine

(13) Ser: Serine

(14) Thr: Threonine

(15) Asp: Aspartic acid

(16) Glu: Glutamic acid

(17) Asn: Asparagine

(18) Gln: Glutamine

(19) Cys: Cysteine

(20) Pro: Proline

(21) Orn: Ornithine

(22) Sar: Sarcosine

(23) Cit: Citrulline

(24) N-Val: Norvaline

(25) N-Leu: Norleucine

(26) Abu: α-Aminobutyric acid

(27) Tau: Taurine

(28) Hyp: Hydroxyproline

(29) t-Leu: tert-Leucine

Furthermore, the amino acid derivative can represent various derivatives of the above-described amino acids, and examples thereof include an unusual amino acid, a non-natural amino acid, an amino alcohol, and an amino acid in which an amino acid side chain such as the terminal carbonyl group, the terminal amino group, or the thiol group of cysteine is replaced with various substituents. Examples of the substituents include an alkyl group, an acyl group, a hydroxy group, an amino group, an alkylamino group, a nitro group, a sulfonyl group, and various protection groups. Examples of the substituted amino acid include: Arg(NO₂): N-γ-nitroarginine; Cys(SNO): S-nitrocysteine; Cys(S-Me): S-methylcysteine; Cys(S-allyl): S-allylcysteine; Val-NH₂: valinamide; and Val-ol: valinol (2-amino-3-methyl-1-butanol).

It should be noted that the “(O)” in γ-Glu-Met(O) and γ-Glu-Cys(S-Me)(O) indicates a sulfoxide structure. The “γ:gamma” in γ-Glu indicates that the glutamic acid binds to another amino acid via the carboxy group at the γ position of the glutamic acid.

In the peptide, “X” can be Cys, Cys(SNO), Cys(S-allyl), Gly, Cys(S-Me), Abu, or Ser, and “Y” can be Gly, Val, Glu, Lys, Phe, Ser, Pro, Arg, Asp, Met, Thr, His, Orn, Asn, Cys, or Gln. However, “X” and “Y” are not limited thereto. Another example of the peptide is γ-Glu-Val-Gly.

A commercially available product can be used as the above-described peptide. Furthermore, the peptide can be obtained by appropriately employing a known technique such as (1) a chemical synthesis method or (2) a synthesis method through an enzymatic reaction. The peptide can contain 2 to 3 amino acid residues, that is, is relatively short, and hence, the chemical synthesis method is convenient. In the case of the chemical synthesis, the oligopeptide can be synthesized or semi-synthesized by using a peptide synthesizer. An example of the chemical synthesis method is a peptide solid phase synthesis method. The peptide synthesized as described above can be purified by general means such as ion exchange chromatography, reversed phase high performance liquid chromatography, or affinity chromatography. Such peptide solid phase synthesis method and the subsequent peptide purification are well known in the technical field.

Furthermore, the peptide can also be produced by an enzymatic reaction. For example, the method described in WO 2004/011653 A1 can be employed. That is, the peptide can also be produced by reacting one amino acid or dipeptide with an esterified or amidated carboxyl terminus with an amino acid having a free amino group (for example, an amino acid whose carboxyl group is protected) in the presence of a peptide-producing enzyme, and purifying the produced a dipeptide or tripeptide.

The peptide-producing enzyme can be a part of a composition which includes a culture of a microorganism having an ability to produce the peptide, microbial cells which have been separated from such a culture, and a processed product of these microbial cells. The peptide-producing enzyme can also be purified or derived from the microorganism which produces it.

It should be noted that the peptide can not only be produced by such enzymatic chemical synthesis methods, but also can also be derived from, for example, a plant such as a vegetable or a fruit, a microorganism such as a yeast, and a yeast extract. When the peptide is native to a natural product, the peptide can be extracted from the product.

Furthermore, the peptide does not need to be isolated before use, and a fraction containing the peptide in a large amount can also be used. An example includes a yeast extract containing glutathione (γ-Glu-Cys-Gly) or a fraction thereof. The preparation of the yeast extract or the like can be conducted in the same manner as general yeast extract preparation. The yeast extract can be a treated product of yeast cells extracted with hot water, or can be a treated product of digested yeast cells. The fraction of the yeast extract is not particularly limited as long as the fraction contains glutathione.

The compound represented by the chemical formula (1) and the compound represented by the chemical formula (2) can be synthesized by, for example, a known method, such as that described in U.S. Pat. No. 6,211,244 A. Furthermore, a commercially available product thereof can also be used.

WO 2007/055388, WO 2008/139945, WO 2008/139946, WO 2008/139947 each describe that the above-described amino acids and peptides are able to activate CaSR.

In addition, examples of CaSR activators include compounds described in U.S. Pat. No. 6,211,244, WO 06/123725, WO 05/115975, U.S. Pat. No. 6,313,146, U.S. Pat. No. 6,213,146, U.S. Pat. No. 5,688,938, U.S. Pat. No. 5,763,569, U.S. Pat. No. 5,858,684, U.S. Pat. No. 5,962,314, U.S. Pat. No. 6,001,884, U.S. Pat. No. 6,011,068, U.S. Pat. No. 6,031,003, WO 95/11221, WO 1996/012697, WO 2002/059102, and JP 1999-130737 A.

Furthermore, another example of a CaSR activator is cinacalcet. Cinacalcet can be synthesized by a known method, and a commercially available product thereof can also be used.

Furthermore, examples of the CaSR activator include the following compounds:

(R)—N-[3-(4-methylphenyl)prop-2-enyl]-1-(3-methoxyphenyl)ethylamine,
(R)—N-[3-(2-methylphenyl)prop-2-enyl]-1-(3-methoxyphenyl)ethylamine,
(R)—N-[3-(2,4,6-trimethylphenyl)prop-2-enyl]-1-(3-methoxyphenyl)ethylamine,
(R)—N-[3-(4-isopropylphenyl)prop-2-enyl]-1-(3-methoxyphenyl)ethylamine,
(R)—N-[3-(2,4-dimethylphenyl)prop-2-enyl]-1-(3-methoxyphenyl)ethylamine
(R)—N-[3-(3-methylphenyl)prop-2-enyl]-1-(3-methoxyphenyl)ethylamine,
(R)—N-(2-methyl-3-phenylprop-2-enyl)-1-(3-methoxyphenyl)ethylamine,
(R,R)—N-(2-methyl-4-phenylbut-3-enyl)-1-(3-methoxyphenyl)ethylamine,
(S,R)—N-(2-methyl-4-phenylbut-3-enyl)-1-(3-methoxyphenyl)ethylamine,
(R)—N-[3-(3-trifluoromethoxyphenyl)propyl]-1-(3-methoxyphenyl)ethylamine,
(R)—N—[(R)-3-(3-trifluoromethylphenyl)-1-methylpropyl]-1-(3-methoxyphenyl)ethylamine,
(R)—N—[(R)-3-(3-trifluoromethoxyphenyl)-1-methylpropyl]-1-(1-naphthyl)ethylamine).

The composition which promotes bicarbonate secretion can contain the CaSR activator as an active ingredient. The composition can contain just one CaSR activator, or can contain two or more CaSR activators.

When using a mixture of CaSR activators, if the CaSR activator is a peptide or a low-molecular-weight compound, the other CaSR present in the mixture can be a cation such as calcium and gadolinium. Calcium is a particular example.

A part of or all of the CaSR activator can also be used in the form of a salt as well as a free compound. Therefore, the term “CaSR activator” can include both the free compound and a salt thereof. Examples of the salt form include acid addition salts and salts with a base, and a salt which is acceptable for use in a pharmaceutical or a food can particularly be used. Such salt forms include, for example, an inorganic salt such as a hydrochloride, a hydrobromide, a sulfate, or a phosphate, or an organic salt such as an acetate, a lactate, a citrate, a tartrate, a maleate, a fumarate, or a monomethyl sulfate.

The composition which promotes bicarbonate secretion can function in the gastrointestinal tract. For example, the gastrointestinal tract includes the stomach and small intestine, including the duodenum, jejunum, and ileum. In particular, the composition remarkably promotes bicarbonate secretion in the stomach and duodenum. The composition which promotes bicarbonate secretion can be used as a pharmaceutical for the treatment or prophylaxis of acid secretion-related diseases, for example. The treatment described herein includes improving the conditions or symptoms and the prophylaxis of progression (exacerbation) of conditions or symptoms related to acid-secretion diseases.

Examples of the “acid secretion-related diseases” include diseases which are induced by the exposure to gastric acid, a digestive enzyme, or a drug such as NSAID in the esophagus, stomach, and small intestine (duodenum, jejunum, and ileum), and which can be treated or prevented by promoting bicarbonate secretion. Examples of the acid secretion-related diseases include a gastric ulcer, duodenal ulcer, NSAID-induced ulcer, GERD, and NERD. The composition which promotes bicarbonate secretion can be particularly used for the treatment or prophylaxis of NSAID-induced ulcers. γ-Glu-Val-Gly and cinacalcet can be used for the treatment or prophylaxis of NSAID-induced ulcer. In the treatment, a therapeutic or prophylactic composition can be used for treatment or prophylaxis of NSAID-induced ulcers including the active ingredient γ-Glu-Val-Gly or cinacalcet

The composition which promotes bicarbonate secretion is typically more effective when used in combination with various medicaments which inhibit aggressive factors, such as a histamine H2 receptor antagonist (H2 blocker) or a proton pump inhibitor. Examples of the histamine H2 receptor antagonist include, but are not limited to, cimetidine, ranitidine, famotidine, and nizatidine, and examples of the proton pump inhibitor include, but are not limited to, omeprazole, lansoprazole, and rabeprazole.

Furthermore, as described above, the CaSR activator can promote somatostatin secretion. Thus, a composition which promotes somatostatin secretion containing the CaSR activator as the active ingredient can be used. Somatostatin is known to inhibit gastric acid secretion, for example. Accordingly, the somatostatin secretion promoter can be used effectively to attenuate aggressive factors.

The somatostatin secretion promoter can also be used as an appetite inhibitor. Thus, the somatostatin secretion promoter and an appetite stimulator can be used in combination to regulate appetite. The “appetite regulation” can refer to enhancing the appetite of people with anorexia, or promoting normal eating behavior in people who tend to overeat. The appetite regulator can be used for the treatment or prophylaxis of conditions such as these.

The above-described appetite regulator composition includes, as components, the somatostatin secretion promoter and the appetite stimulator, and one of these components can be alternatively selected during use. That is, when appetite inhibition is needed, the somatostatin secretion promoter is used, and when appetite stimulation is needed, the appetite stimulator is used, before administration or ingestion. An example of an appetite stimulator is sodium glutamate.

The composition which promotes bicarbonate secretion or regulates appetite can be useful for mammals, such as mice, rats, hamsters, rabbits, cats, dogs, cattle, sheep, monkeys, and human beings.

There are no particular limitations on the dosage form or the type of administration of the composition which promotes bicarbonate secretion, except that it contains the CaSR activator. The administration can be oral or parenteral administration (ingestion), such as via drip infusion or injection (transveous administration). Oral administration is typically the easiest and most convenient method, but the administration is not limited thereto.

General dosage forms for orally-administered pharmaceutical formulations include a granule, a fine granule, a dust formulation, a coated tablet, a tablet, a suppository, a powder, a (micro)capsule, a chewable, a syrup, a juice, a solution, a suspension, and an emulsion. Formulations appropriate for direct intravenous infusion, drip infusion, and controlled- or delayed-release can be used when administering via injection.

When administering orally, the dosage can be any amount as long as it is effective for treatment and/or prophylaxis, although an appropriate dosage can vary depending on the symptoms and age of a patient. The dosage can be appropriately adjusted depending on the patient's age, gender, body weight, symptoms, and the like. For example, in the case of oral administration, the dosage can be 0.001 g to 10 g/kg body weight per day for an adult, and in another example, 0.1 g to 1 g/kg body weight per day for an adult, in terms of the amount of the CaSR activator.

Furthermore, the dosage in the case of parenteral administration such as drip infusion or injection (transvenous administration) can be about 10- to 20-fold less than the dosage in the exemplary range for the oral administration (ingestion amount).

The above-described dosage for oral administration can be similarly applied to a food as well. However, the CaSR activator can be included in the food so that the amount ingested is smaller than the above-described dosage in the oral administration.

The composition which promotes bicarbonate secretion or regulates appetite can be formulated by a conventional method. If required for formulation, various pharmacologically acceptable substances can be blended. Such substances can be appropriately selected depending on the dosage form of the formulation, and examples of the substances for formulation include an excipient, a diluent, an additive, a disintegrant, a binder, a coating agent, a lubricating agent, a sliding agent, a lubricant, a flavoring agent, a sweetener, and a solubilizer. In addition, specific examples of the substances for formulation include magnesium carbonate, titanium dioxide, lactose, mannitol, and other sugars, talc, cow's milk protein, gelatin, starch, cellulose and its derivatives, animal and vegetable oils, polyethylene glycol, and solvents such as sterile water and monohydric or polyhydric alcohols such as glycerol. It should be noted that, in the appetite regulator, the somatostatin secretion promoter and the appetite stimulator are each individually formulated and packaged separately.

The composition which promotes bicarbonate secretion or regulates appetite can be formulated not only by a conventional method, but also by various pharmaceutical formulation forms to be developed in the future. To the formulation, a method to be developed in the future can be appropriately adopted.

A document with instructions for use can be inserted in a package which includes the composition(s). An example of the document is a so-called a package insert with instructions for usage, efficacy, administration method, and the like.

The composition which promotes bicarbonate secretion can be incorporated into a food. The form of the food is not particularly limited, and the food can be manufactured by the same method using the same materials by which it is normally manufactured, except that the CaSR activator is blended into it. Exemplary foods include seasonings, beverages such as juice or cows milk, confectioneries, jellies, health foods, processed agricultural products, processed fishery products, processed animal products such as cows milk, and food supplements. Furthermore, foods containing the bicarbonate secretion promoter can be marketed and sold with health claims, which means the food packaging can include a label indicating that the food can be used for the prophylaxis, treatment, or improvement of acid secretion-related diseases, and in particular, can be used with foods for specified health uses and the like.

The composition which promotes bicarbonate secretion can be prepared in the form of a tablet, a capsule, a powder, a granule, a suspension, a chewable, a syrup, or the like. The food supplement can refer to a product which is ingested for the purpose of supplementing nutrients as well as ingested as a food, and also includes a nutritious supplement, a supplement, and the like. Furthermore, the food supplement can be a part of foods with health claims.

### EXAMPLES

Hereinafter, the present invention is more specifically described by way of examples. However, the present invention is not limited thereto.

### Production Example 1

### Synthesis of γ-Glu-Val-Gly

Boc-Val-OH (8.69 g, 40.0 mmol) and Gly-OBzl.HCl (8.07 g, 40.0 mmol) were dissolved in methylene chloride (100 ml) and the solution was kept at 0° C. Triethylamine (6.13 ml, 44.0 mmol), HOBt (1-hydroxybenzotriazole, 6.74 g, 44.0 mmol), and WSC.HCl (1-ethyl-3-(3-dimethylaminopropyl)carbodiimide hydrochloride, 8.44 g, 44.0 mmol) were added to the solution, and the mixture was stirred overnight at room temperature. The reaction solution was concentrated under reduced pressure, and the residue was dissolved in ethyl acetate (200 ml). The solution was washed with water (50 ml), a 5% citric acid aqueous solution (50 ml×twice), saturated saline (50 ml), a 5% sodium bicarbonate aqueous solution (50 ml×twice), and saturated saline (50 ml). The organic layer was dried over anhydrous magnesium sulfate, magnesium sulfate was removed by filtration, and the filtrate was concentrated under reduced pressure. The residue was recrystallized from ethyl acetate-n-hexane to yield Boc-Val-Gly-OBzl (13.2 g, 36.2 mmol) as a white crystal. Boc-Val-Gly-OBzl (5.47 g, 15.0 mmol) was added to a 4 N HCl/dioxane solution (40 ml), and the mixture was stirred at room temperature for 50 minutes. Dioxane was removed by concentration under reduced pressure, n-hexane (30 ml) was added to the residue, and the mixture was concentrated under reduced pressure. The procedure was repeated three times to quantitatively provide H-Val-Gly-OBzl.HCl.

H-Val-Gly-OBzl.HCl described above and Z-Glu-OBzl (5.57 g, 15.0 mmol) were dissolved in methylene chloride (50 ml), and the solution was kept at 0° C. Triethylamine (2.30 ml, 16.5 mmol), HOBt (1-hydroxybenzotriazole, 2.53 g, 16.5 mmol), and WSC.HCl (1-ethyl-3-(3-dimethylaminopropyl)carbodiimide hydrochloride, 3.16 g, 16.5 mmol) were added to the solution, and the mixture was stirred at room temperature overnight for 2 days. The reaction solution was concentrated under reduced pressure, and the residue was dissolved in heated ethyl acetate (1500 ml). The solution was washed with water (200 ml), a 5% citric acid aqueous solution (200 ml×twice), saturated saline (150 ml), a 5% sodium bicarbonate aqueous solution (200 ml×twice), and saturated saline (150 ml). The organic layer was dried over anhydrous magnesium sulfate, magnesium sulfate was removed by filtration, and the filtrate was concentrated under reduced pressure. The precipitated crystal was collected by filtration and dried under reduced pressure to yield Z-Glu(Val-Gly-OBzl)-OBzl (6.51 g, 10.5 mmol) as a white crystal.

Z-Glu(Val-Gly-OBzl)-OBzl described above (6.20 g, 10.03 mmol) was suspended in ethanol (200 ml), 10% palladium/carbon (1.50 g) was added to the suspension, and a reduction reaction was performed under a hydrogen atmosphere at 55° C. for 5 hours. During the reaction, 100 ml in a total volume of water was gradually added. The catalyst was removed by filtration using a Kiriyama funnel, and the filtrate was concentrated under reduced pressure to a half volume. The reaction solution was further filtered through a membrane filter, and the filtrate was concentrated under reduced pressure. The residue was dissolved in a small volume of water, and then ethanol was added to precipitate a crystal. The crystal was collected by filtration and dried under reduced pressure to yield γ-Glu-Val-Gly as a white powder (2.85 g, 9.40 mmol). Hereinafter, γ-Glu-Val-Gly is also referred to as “γ-EVG.”

ESI-MS: (M+H)⁺=304.1.

¹H-NMR (400 MHz, D₂O) δ (ppm): 0.87 (3H, d, J=6.8 Hz), 0.88 (3H, d, J=6.8 Hz), 1.99-2.09 (3H, m), 2.38-2.51 (2H, m), 3.72 (1H, t, J=6.35 Hz), 3.86 (1H, d, J=17.8 Hz), 3.80 (1H, d, J=17.8 Hz), 4.07 (1H, d, J=6.8 Hz).

### Production Example 2

### Synthesis of (R)—N-(3-(3-trifluoromethylphenyl)propyl)-1-(1-naphthyl)ethylamine hydrochloride (cinacalcet hydrochloride)

### Step 1: Synthesis of 3-(3-trifluoromethylphenyl)-propionic acid methyl ester

A mixture of 2.20 g of 3-(trifluoromethyl)cinnamic acid, 166 mg of palladium/carbon (10%, wet), and 40 ml of ethanol was stirred overnight under a hydrogen atmosphere at 1 atm. Palladium/carbon was separated by filtration, and the filtrate was concentrated under reduced pressure. 20 ml of methanol and 4 drops of concentrated sulfuric acid were added and the mixture was stirred at 60° C. for 2 hours, and then left to cool down. After concentration under reduced pressure, 20 ml of a saturated sodium bicarbonate aqueous solution was added, and the resultant was extracted with 20 ml of dichloromethane. The extract was dried over anhydrous sodium sulfate and then concentrated under reduced pressure to afford 2.40 g of the captioned compound as an oil.

¹H-NMR (300 MHz, CDCl₃) δ 2.66 (2H, t, J=7.5 Hz), 3.02 (2H, t, J=7.5 Hz), 3.68 (3H, s), 7.37-7.50 (4H, m)

### Step 2: Synthesis of 3-(3-trifluoromethylphenyl)propanal

2.40 g of 3-(3-trifluoromethylphenyl)-propionic acid methyl ester synthesized in Step 1 was dissolved in 20 ml of dry dichloromethane. 13 ml of diisopropyl aluminum hydride solution (0.91 M) in hexane was dropped over 5 minutes at −78° C. under an argon atmosphere, and the mixture was stirred at the same temperature for 40 minutes. 50 ml of a saturated ammonium chloride aqueous solution was dropped, and then the mixture was stirred and the temperature was raised to room temperature. 20 ml of water and 5 ml of concentrated hydrochloric acid were added, and the resultant was separated into an aqueous layer and an organic layer. The aqueous layer was extracted with dichloromethane, and the extract was combined with the separated organic layer, dried over anhydrous sodium sulfate, and then concentrated under reduced pressure to afford 2.12 g of the captioned compound as an oil.

¹H-NMR (300 MHz, CDCl₃) δ 2.83 (2H, t-t, J=7.5 Hz, 1.2 Hz), 3.02 (2H, t, J=7.5 Hz), 7.36-7.52 (4H, m), 9.83 (1H, t, J=1.2 Hz)

### Step 3: Synthesis of (R)—N-(3-(3-trifluoromethylphenyl)propyl)-1-(1-naphthyl)ethylamine hydrochloride (cinacalcet hydrochloride)

To a mixture of 2.12 g of 3-(3-trifluoromethylphenyl)propanal synthesized in Step 2, 2.0 ml of (R)-1-(1-naphthyl)ethylamine, 3.42 g of sodium triacetoxyborohydride, and 150 ml of dry dichloromethane was added 0.750 ml of glacial acetic acid, and the mixture was stirred at room temperature for 5 hours. After 100 ml of water had been added and the mixture had been stirred for 3 hours, 100 ml of a 2M sodium hydroxide aqueous solution was added, and the resultant was separated into an aqueous layer and an organic layer. The aqueous layer was extracted with dichloromethane, and the extract was combined with the separated organic layer, dried over anhydrous sodium sulfate, and then concentrated under reduced pressure. The residue was purified with column chromatography (silica gel/hexane:ethyl acetate of 4:1 to 1:1) and then concentrated to afford 3.41 g of (R)—N-(3-(3-trifluoromethylphenyl)propyl)-1-(1-naphthyl)ethylamine as an oil. The compound was dissolved in 10 ml of dichloromethane, 5 ml of 4M hydrochloric acid/dioxane and 20 ml of toluene were added, and the mixture was concentrated under reduced pressure to dryness. The residue was recrystallized from 40 ml of ethanol and 200 ml of heptane to afford 1.71 g of the captioned compound.

¹H-NMR (300 MHz, DMSO-d⁶) δ 1.69 (3H, d, J=6.6 Hz), 2.00 (2H, quintet, J=7.8 Hz), 2.72 (2H, t, J=7.5 Hz), 2.65-2.85 (1H, br), 2.90-3.05 (1H, br), 5.24-5.38 (1H, br), 7.44-7.67 (7H, m), 7.96-8.04 (3H, m), 8.23-8.28 (1H, pseud d), 9.20-9.40 (1H, br), 9.80-10.00 (1H, br)

MS (ESI, m/z) 358 (MH⁺)

### Example 1

### Effect of Cinacalcet on Bicarbonate Secretion in Stomach and Duodenum

The bicarbonate secretion was evaluated in accordance with the methods of Aihara et al. (Aihara, E. et al. 2005. J. Pharmacol. Exp. Ther. 315: 423-432) and Kagawa et al. (Kagawa, S. et al. 2003. Digestive Diseases and Sciences 48: 1850-1856).

The bicarbonate secretion in the stomach was measured as described below. The abdomen of each of male SD rats was opened in the midline under anesthesia with urethane (1.25 g/kg/5 ml, intraperitoneal administration) to expose the stomach. The stomach was left to stand still in an ex-vivo chamber (Tamura Seisakusho; 3.1 cm²) and perfused with physiological saline saturated with 100% O₂ gas. In order to inhibit acid secretion, 60 mg/kg omeprazole was intraperitoneally administered. The bicarbonate secretion was continuously measured by dropping 2 mM HCl to a perfusate until an end point of pH 7.0 by employing a pH-stat method (TOA, AUT-501).

As for the bicarbonate secretion in the duodenum, the abdomen of each of male SD rats was opened under anesthesia, and a duodenum loop having a length of 1.7 cm from the pyloric ring of the stomach was prepared. The lumen of the loop was perfused with physiological saline saturated with 100% O₂ gas (flow rate: 1 ml/min)

A drug was administered into the gastric or duodenal lumen after the bicarbonate secretion at the baseline had been measured for 30 minutes, and the bicarbonate secretion amount was measured every 10 minutes.

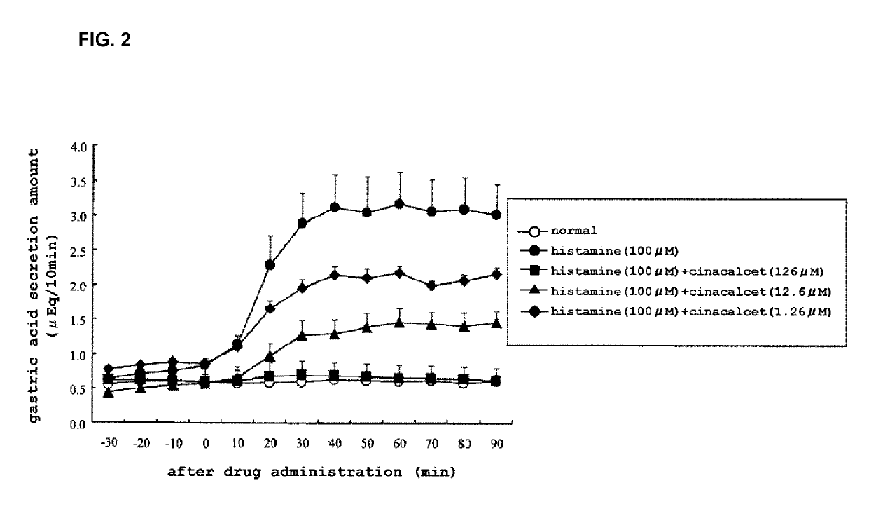

FIG. 2 illustrates the results. Cinacalcet (10 mg/kg) promoted bicarbonate secretion in the stomach and duodenum.

### Example 2

### Influence of Cinacalcet on Histamine-Induced Gastric Acid Secretion

A model prepared in accordance with the method reported by Horie et al. (British Journal of Pharmacology 1994; 112: 87-92) was used as a model for histamine-induced gastric acid secretion. That is, 6-week-old male ddY mice were fasted for 18 hours, and the stomach was removed from each of the mice under anesthesia with urethane (1.25 g/kg/10 ml, intraperitoneal administration). A polyethylene cannula was inserted at each of the pyloric and esophageal sides of the stomach, and the stomach was left to stand still in an organ bath filled with a buffer (118.1 mM NaCl, 4.8 mM KCl, 1.0 mM KH₂PO₄, 16.0 mM Na₂HPO₄, 1.2 mM MgSO₄, 0.65 mM CaCl₂, and 31.6 mM glucose, saturated with 95% O₂/5% CO₂ gas, 37° C.). The intragastric pressure was kept at 15 cmH₂O, and the lumen of the stomach was perfused with a buffer (135.8 mM NaCl, 4.8 mM KCl, 1.2 mM MgSO₄, 1.3 mM CaCl₂, and 31.6 mM glucose, pH 5.4, saturated with 95% O₂/5% CO₂ gas, 37° C.) at a flow rate of 1 ml/min

The acid secretion amount was continuously measured by dropping 10 mM NaOH to a perfusate until an end point of pH 5.4 by employing a pH-stat method (TOA, AUT-501). The intragastric acid secretion amount during no stimulation was measured for 30 minutes, and then the remove stomach left to stand still in the organ bath was treated with 100 μM histamine and cinacalcet (1.26, 12.6, and 126 μM). The gastric acid secretion amount was titrated for up to 90 minutes. The Dunnett's test was used as a statistical test for an effect of cinacalcet.

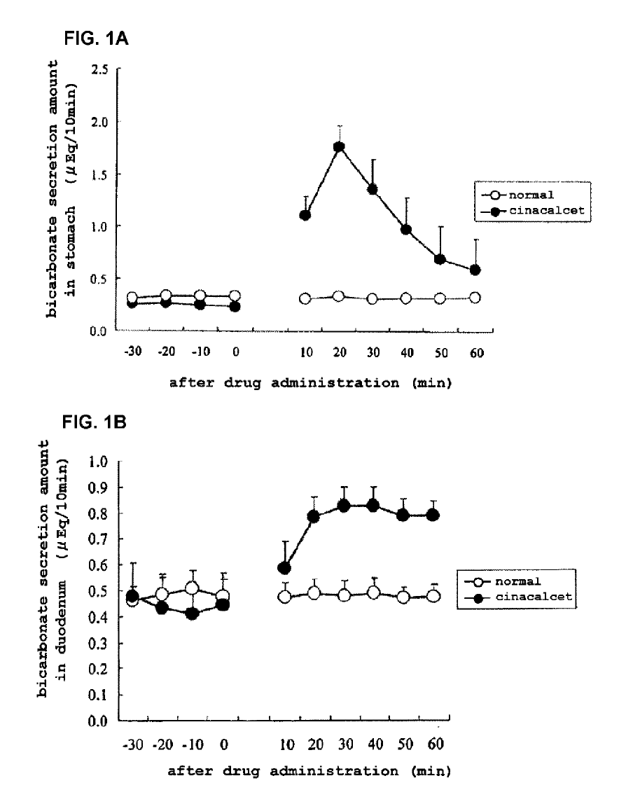

FIG. 1 illustrate the results. The acid secretion amount, which was 0.5 to 0.8 nEq/10 min during no stimulation, increased to about 3.2 nEq/10 min by histamine stimulation. Cinacalcet inhibited histamine-induced gastric acid secretion dose-dependently, and completely inhibited gastric acid secretion at a concentration of 126 μM.

### Example 3

### Expression of Somatostatin and CaSR in Fractions of Rat Gastric Mucosal D Cells

The abdomen of each male SD rat was opened in the midline under anesthesia to remove the stomach. The stomach was ligated at the cardiac region and at the junction between the gastric body and the pylorus, and the pyloric region was excised. After that, the stomach was everted so that the luminal side is exposed outside. To the inside of the stomach, Buffer A containing 2.5 mg/ml protease (Sigma P6911, Protease, From Streptomyces griseus, 5.5 unit/mg solid) was injected to prepare a balloon. The balloon was shaken at 37° C. for 30 minutes in Buffer A for 30 minutes and in Buffer B for 30 minutes×three times. After that, the balloon was stirred three times in Buffer B containing 0.5 mg/ml DNase, the detached cells were centrifuged and then suspended in Buffer C. After the cell suspension had been filtered with a filter, cells were fractionated and collected with an elutriator manufactured by Hitachi, Ltd.

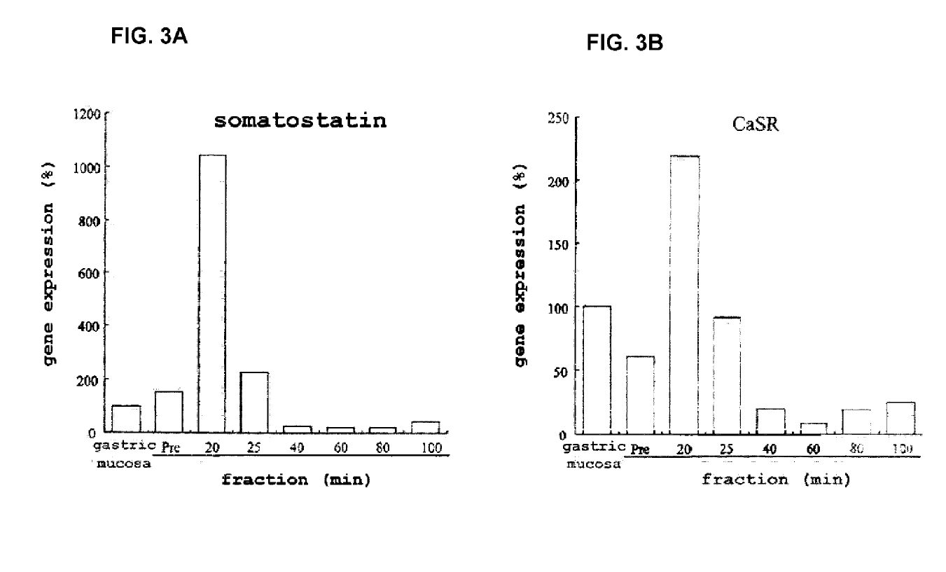

The mRNA expression amounts of somatostatin and CaSR in the above-described cell fractions and the entire gastric mucosa were measured by a real-time RT-PCR method. FIG. 3 illustrate the mRNA expression amounts of somatostatin and CaSR in each of the above-described cell fractions in terms of relative values (gene expression (%)) to the mRNA expression amounts in the entire gastric mucosa. The results revealed that one part (20 ml/min) of the cell fractions highly expressed somatostatin and CaSR in an extremely specific manner as compared to the other parts of the cell fractions. Accordingly, it was considered that the cells fractionated under this condition were mainly D cells as somatostatin-producing cells, and the D cells expressed CaSR.

The composition of each of the buffers is as follows:

Buffer A (70 mM NaCl, 5 mM KCl, 11 mM glucose, 50 mM HEPES, 0.5 mM NaH₂PO₄, 1.0 mM Na₂HPO₄, 20 mM NaHCO₃, 2 mM Na₂EDTA, 20 mg/ml BSA); Buffer B (70 mM NaCl, 5 mM KCl, 1.5 mM MgSO₄, 1.0 mM CaCl₂, 11 mM glucose, 50 mM HEPES, 0.5 mM NaH₂PO₄, 1.0 mM Na₂HPO₄, 20 mM NaHCO₃, 20 mg/ml BSA);

Buffer C (70 mM NaCl, 5 mM KCl, 1.5 mM MgSO₄, 1.0 mM CaCl₂, 11 mM glucose, 50 mM HEPES, 0.5 mM NaH₂PO₄, 1.0 mM Na₂HPO4, 20 mM NaHCO₃, 0.5 mM dithiothreitol, 1 mg/ml BSA).

### Example 4

### Somatostatin Secretion in the Case of Treating Fractionated D Cells with CaSR Agonist

Repeated fractionation was conducted using an elutriator in accordance with Example 3 to collect D cells with high purity. After centrifugation, the cells were suspended in a cell culture medium (D-MEM/F-12 containing 10% FBS) and cultured in a CO₂ incubator (95% O₂+5% CO₂, 37° C.) (6×10⁵ cells/250 μL/well). After 2-day culture, to the D cells washed with PBS was added each of CaSR agonists (20 μM cinacalcet (CCT), 20 μM γ-EVG, 10 mM phenylalanine (Phe), and 10 mM histidine (His)) mixed in a culture buffer (122 mM NaCl, 5 mM KCl, 25 mM NaHCO₃, 1.3 mM MgSO₄, 2 mM CaCl₂, 15 mM HEPES, and 20 mM glucose, pH 7.4), and the cells were cultured for 90 minutes. After that, a culture supernatant was collected, filtered with a filter, and then measured for its somatostatin concentration with a somatostatin ELISA kit (manufactured by Phoenix, Calif.).

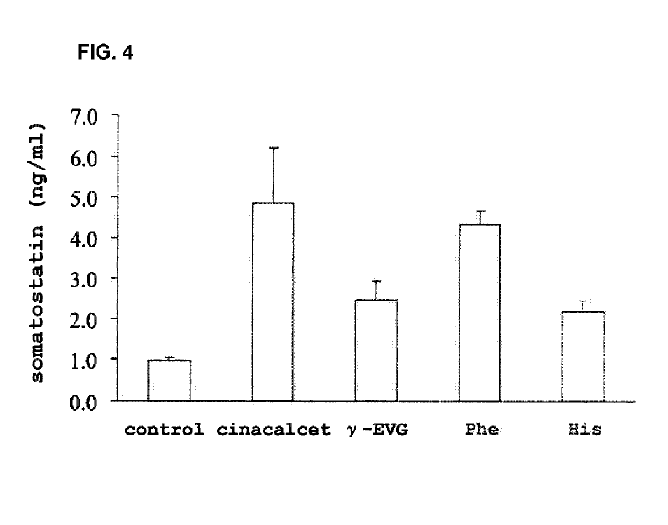

FIG. 4 illustrates the results. In the cultured D cells, cinacalcet, γ-EVG, Phe, and His each remarkably promoted somatostatin secretion.

### Example 5

### Gastric Acid Secretion in the Case of Using Cinacalcet and Somatostatin Receptor 2 Inhibitor in Combination for Histamine-Induced Gastric Acid Secretion

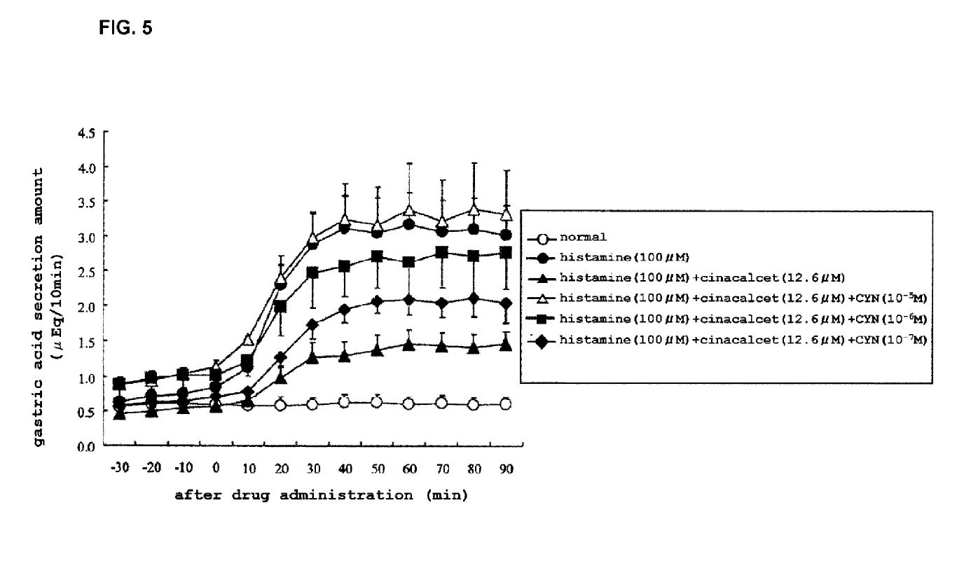

The gastric acid secretion was measured in accordance with the method of Example 2. FIG. 5 illustrates the results. 100 μM histamine promoted gastric acid secretion and the effect was inhibited by 12.6 μM cinacalcet. When the removed stomach left to stand still in an organ bath was treated with histamine, cinacalcet, and CYN154806 (SIGMA) (10⁻⁷ to 10⁻⁵ M) as an inhibitor selective to somatostatin receptor 2, the inhibition of gastric acid secretion with cinacalcet was released depending on the dose of CYN154806, resulting in the enhancement of gastric acid secretion. This suggested that cinacalcet affected somatostatin secretion to inhibit gastric acid secretion.

### Example 6

### Effect of Cinacalcet or γ-EVG on Non-Steroidal Anti-Inflammatory Drug (NSAID)-Induced Enteritis

To non-fasting rats, distilled water for injection containing cinacalcet (1, 3, 10, and 100 mg/kg) or γ-EVG (100 mg/kg), or distilled water for injection (control) was orally administered. After 30 minutes, loxoprofen (60 mg/kg) was orally administered, and the rats were left to stand for 24 hours. 1 ml of 1% (w/w) Evans Blue dye was intravenously administered, and after 30 minutes, the rats were euthanized under deep ether anesthesia. The small intestine (from the duodenum to the ileum) of each of the rats was removed, then immersed in 2% formalin for 10 minutes so that the small intestine might be fixed from the serosal side, and incised from the opposite side of the mesentery, and the damaged area in the small intestine (mm²) was measured with a dissecting microscope of 10 times power. The t-test or Dunnett's test was used for a statistical test and p<0.05 was considered significant.

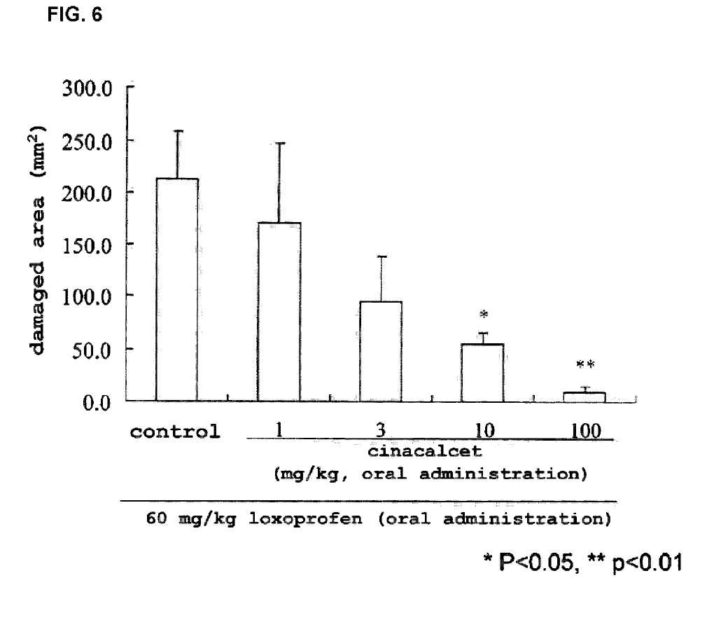

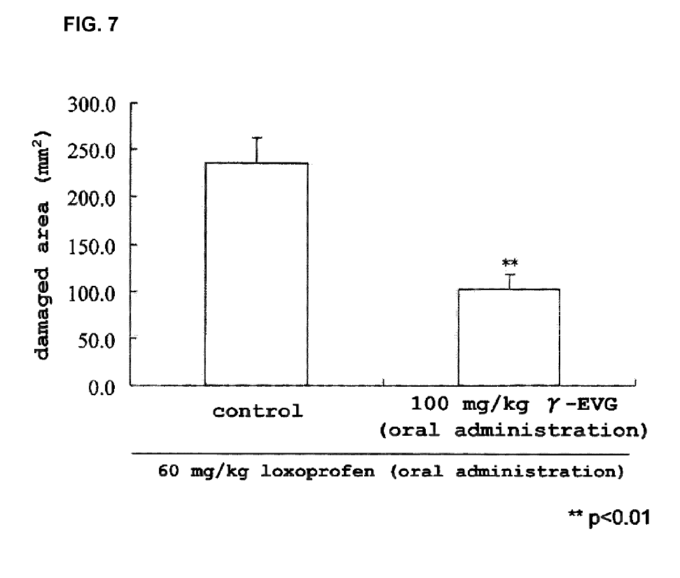

FIGS. 6 and 7 illustrate the results. Cinacalcet reduced the damaged area dose-dependently. Furthermore, γ-EVG also reduced the damaged area. That is, the results showed that those medicaments as calcium receptor activators each had a preventive effect on NSAID-induced enteritis. It is understood that those medicaments are useful for the prophylaxis or treatment of NSAID-induced ulcer.

### Example 7

### Influence of γ-EVG on Bicarbonate Secretion in Duodenum

The abdomens of male SD rats were opened under anesthesia, and a duodenum loop having a length of 1.7 cm from the pyloric ring of the stomach was prepared. The lumen of the loop was perfused with physiological saline saturated with 100% O₂ gas (flow rate: 1 ml/min).

After the bicarbonate secretion at the baseline had been measured for 30 minutes, distilled water for injection containing γ-EVG (100 mg/kg) or distilled water for injection (normal) was administered into the duodenal lumen, and the bicarbonate secretion amount was measured every 10 minutes.

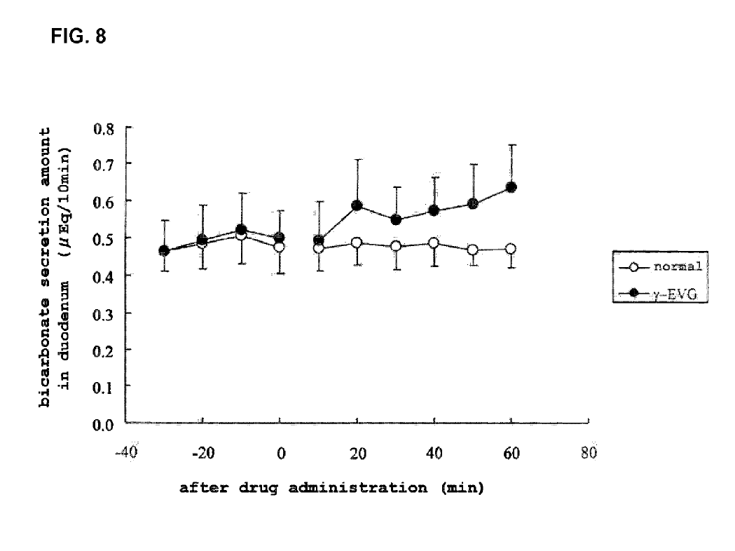

FIG. 8 illustrates the results. γ-EVG as a calcium receptor activator promoted bicarbonate secretion in the duodenum.

It is clear from the foregoing that the CaSR activator is effective for the promotion of bicarbonate secretion and for the treatment or prophylaxis of acid secretion-related diseases, for example.

### Example 8

### Somatostatin Secretion in the Case of Treating Fractionated Gastric Mucosal D Cells with CaSR Agonist and Sodium Glutamate

The somatostatin secretion was measured in accordance with the method of Example 4. After 2-day culture, to the D cells washed with PBS was added a CaSR agonist (20 μM γ-EVG), sodium glutamate (60 mM MSG), or a mixed solution thereof, mixed in a culture buffer (122 mM NaCl, 5 mM KCl, 25 mM NaHCO₃, 1.3 mM MgSO₄, 2 mM CaCl₂, 15 mM HEPES, and 20 mM glucose, pH 7.4), and the cells were cultured for 90 minutes. After that, the culture supernatant was collected, filtered with a filter, and then the somatostatin concentration was measured by a somatostatin ELISA kit (Phoenix, Calif.).

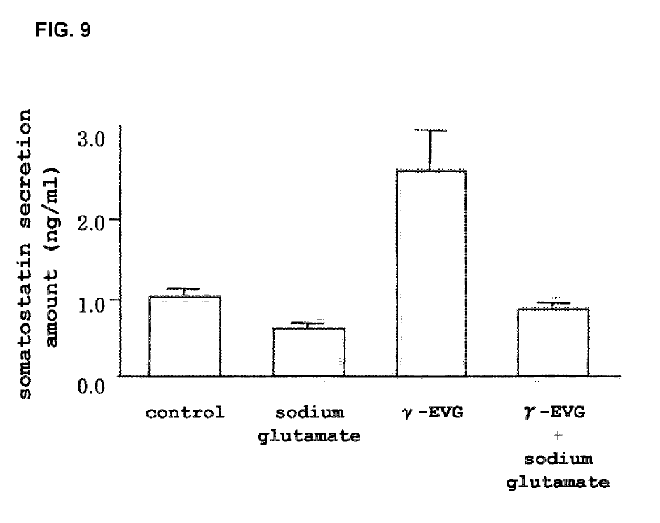

FIG. 9 illustrates the results. In the cultured D cells, γ-EVG markedly promoted somatostatin secretion. On the other hand, MSG remarkably inhibited somatostatin secretion. The mixed solution of γ-EVG and MSG inhibited somatostatin secretion.

### INDUSTRIAL APPLICABILITY

The present invention provides a bicarbonate secretion promoter in the gastrointestinal tract. The bicarbonate secretion promoter in the gastrointestinal tract of the present invention can be utilized in a pharmaceutical or food for the prophylaxis or treatment or improvement of acid secretion-related diseases, particularly gastric ulcers, duodenal ulcers, NSAID-induced ulcers, GERD, NERD, and the like. The pharmaceutical or food of the present invention is highly safe.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

What is claimed is:

1. A method for promoting bicarbonate secretion in the gastrointestinal tract, comprising administering a composition comprising a calcium receptor activator to a subject in need of the promotion of bicarbonate secretion in the gastrointestinal tract.

2. The method according to claim 1, wherein the calcium receptor activator is a peptide.

3. The method according to claim 2, wherein the peptide is selected from the group consisting of γ-Glu-X-Gly, γ-Glu-Val-Y, γ-Glu-Ala, γ-Glu-Gly, γ-Glu-Cys, γ-Glu-Met, γ-Glu-Thr, γ-Glu-Val, γ-Glu-Orn, Asp-Gly, Cys-Gly, Cys-Met, Glu-Cys, Gly-Cys, Leu-Asp, γ-Glu-Met(O), γ-Glu-γ-Glu-Val, γ-Glu-Val-NH₂, γ-Glu-Val-ol, γ-Glu-Ser, γ-Glu-Tau, γ-Glu-Cys(S-Me)(O), γ-Glu-Leu, γ-Glu-Ile, γ-Glu-t-Leu, and γ-Glu-Cys(S-Me), wherein X is selected from the group consisting of an amino acid and an amino acid derivative, and Y is selected from the group consisting of an amino acid and an amino acid derivative.

4. The method according to claim 3, wherein X is selected from the group consisting of Cys, Cys(SNO), Cys(S-allyl), Gly, Cys(S-Me), Abu, and Ser; and Y is selected from the group consisting of Gly, Val, Glu, Lys, Phe, Ser, Pro, Arg, Asp, Met, Thr, His, Orn, Asn, Cys, and Gln.

5. The method according to claim 1, wherein the calcium receptor activator is selected from the group consisting of: 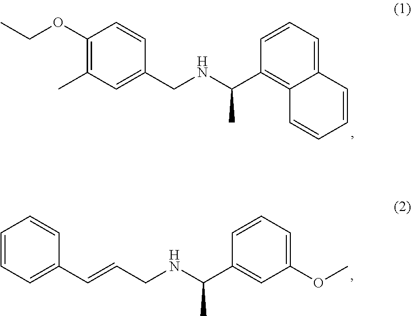
and salts thereof.

6. The method according to claim 2, wherein the peptide is γ-Glu-Val-Gly.

7. The method according to claim 1, wherein the calcium receptor activator is cinacalcet.

8. A method for preventing or treating a disease related to acid secretion comprising administering a composition comprising a calcium receptor activator to a subject in need of the prophylaxis or treatment of an acid secretion-related disease.

9. The method according to claim 8, wherein the disease is selected from the group consisting of gastric ulcer, duodenal ulcer, non-steroidal anti-inflammatory drug-induced ulcer, gastro-esophageal reflux disease, and non-erosive reflux disease.

10. A method for preventing or treating a non-steroidal anti-inflammatory drug-induced ulcer, comprising administering a composition comprising γ-Glu-Val-Gly or a composition comprising cinacalcet to a subject in need of the prophylaxis or treatment of a non-steroidal anti-inflammatory drug-induced ulcer.

11. The method according to claim 8, wherein the calcium receptor activator is administered in an amount of 0.001 to 10 g/kg body weight per day.

12. A method for regulating appetite comprising alternatively administering a composition comprising a calcium receptor activator or a composition comprising an appetite stimulator to a subject in need of appetite regulation.

13. The method according to claim 13, wherein the appetite stimulator is sodium L-glutamate.
